(19) United States
(12) Plant Patent Application Publication
(10) Pub. No.: US 2009/0113584 P1
(43) Pub. Date: Apr. 30, 2009

(54) Vriesea Plant Named 'Kiwi Cream'

(50) ;

(76) Inventor: Andrew Maloy, Whenuapai (NZ)

Correspondence Address: HortisUSA, P.O.Box 3109, Camarillo, CA, 93011, US

(21) Appl. No.: 11/978,482

(22) Filed: Oct. 29, 2007

(51) Int. Cl. A01H 5/00

(52) U.S. Cl. PLT/263.1

(57) ABSTRACT

A new and distinct Vriesea cultivar named ‘Kiwi Cream’ is disclosed, characterized by very stable, strongly reticulate variegated foliage of green/yellow. Upright plant form with a cream heart.

### LATIN NAME OF THE GENUS AND SPECIES

Vriesea lindl.

### VARIETY DENOMINATION

‘Kiwi Cream’

### BACKGROUND OF THE INVENTION

The new cultivar is a product of a planned breeding program. The new variety originated from a cross pollination of an unnamed, unknown seed parent and an unnamed, unknown pollen parent, both Vriesea lindl., made by the inventor during 1996.

It was discovered by the inventor, Andrew Maloy, a citizen of New Zealand, in 1999 in a group of seedlings resulting from the 1996 crossing.

Asexual reproduction of the new cultivar ‘Kiwi Cream’ was first performed in Auckland, New Zealand by tissue culture in 2002. ‘Kiwi Cream’ has been reproduced by micro-propagation and has shown that the unique features of this cultivar are stable and reproduced true to type.

### SUMMARY OF THE INVENTION

The cultivar ‘Kiwi Cream’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Kiwi Cream’ These characteristics in combination distinguish ‘Kiwi Cream’ as a new and distinct Vriesea cultivar:
1. Foliage resistant to stress.
2. Foliage is green/yellow with distinct transverse markings.
3. Paler coloring at center of plant.
4. Suitability as an indoor pot plant.
5. Broad leaves occurring in right, regular whorls.

Plants of the new cultivar ‘Kiwi Cream’ cannot be compared to either seed parent or pollen parent as records of those characteristics were not kept. However, ‘Kiwi Cream’ can be compared to the commercial variety Vriesea fosteriana ‘Red Chestnut.’ Plants of ‘Red Chestnut’ are similar to plants of ‘Kiwi Cream’ in most horticultural characteristics, however, ‘Red Chestnut has a plain, uncolored heart and darker green leaves than ‘Kiwi Cream.’

‘Kiwi Cream’ can also be compared to it seedling siblings, ‘Kiwi Dusk’ and ‘Kiwi Sunset.’ Plant patent applications for these 2 varieties have been filed concurrently with that of ‘Kiwi Cream.’ ‘Kiwi Cream’ differs from ‘Kiwi Dusk’ in having overall much lighter foliage and a lighter center to the plant. ‘Kiwi Cream’ is also normally taller than ‘Kiwi Dusk.’ ‘Kiwi Cream’ differs from ‘Kiwi Sunset’ in having lighter foliage and a center to the plant with cream, rather than pink tones.

### BRIEF DESCRIPTION OF THE PHOTOGRAPH

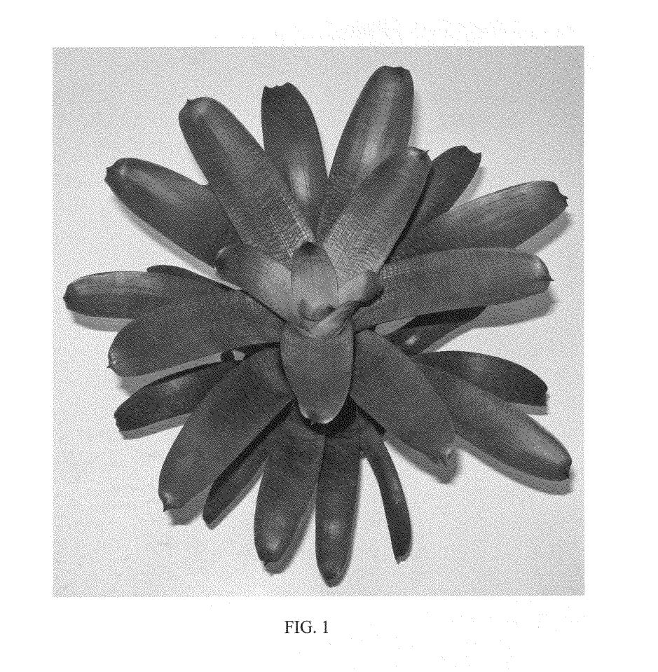

The accompanying photograph in FIG. 1 illustrates in full color a typical plant of ‘Kiwi Cream’ grown in a greenhouse. This plant is approximately 4 years, 6 months old, shown in a 17 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘Kiwi Cream’ plants grown in a climate controlled greenhouse in Saint Oedenrode, the Netherlands. Temperatures ranged from 18° C. to 22° C. at night to 18° C. to 22° C. during the day. No artificial light, photoperiodic treatments or chemical treatments were given to the plants. Measurements and numerical values represent averages of typical plant types.
Botanical classification: Vriesea lindl. cultivar ‘Kiwi Cream.’

### PROPAGATION

Time to rooting: 2 months from a 2 year old tissue culture at approximately 20 to 22° C.
Root description: Fine, fibrous. Free Branching

### PLANT

Growth habit: Upright, forming compact rosettes around a single axis.
Plant shape: Inverted triangle.
Height: Approximately 44.2 cm.
Plant spread: Approximately 40 cm in a 17 cm pot.
Normal pot size: 15 cm and larger.
Growth rate: Slow.
Branching characteristics: Upright, non-branching.
Number of leaves per stem: Approximately 30.
Age of plant described: Approximately 4 years, 6 months.

### FOLIAGE

Leaf:
   Arrangement.—Whorled.
   Average length.—Approximately 29.6 cm.
   Average width.—Approximately 6.6 cm.
   Shape of blade.—Oblong.
   Aspect.—Slightly recurved.
   Apex.—Sharply acute.
   Base.—Decurrent.
   Margin.—Entire.
   Texture of top surface.—Smooth, moderately glossy.
   Texture of bottom surface.—Smooth, moderately glossy.
   Color.—Young foliage upper side: Near R.H.S. Yellow-green; 145A to 145D at the base, darker towards the tip; Upper half of the leaf blade is 144A with yellow-green 146A, 146B and 146C reticulate variegation. Margin at the tip is near greyed-purple; 187A. Young foliage under side. Near R.H.S. Yellow-green 145C/D. Heavily flushed greyed-red towards the tip; 178B. Outer tip greyed-purple; N186C, but slightly lighter Upper half of the leaf blade with a highly mixed, muddy reticulate variegation consisting of; 147A 35C and 199A. Mature foliage upper side. Near R.H.S. Green; 137A/B. Base greyed-green; 192A/B. Outer base greyed-orange; 166A. Upper leaf blade with a green to yellow-green reticulate variegation; Darker than 139A/147A. Tip greyed-purple; 187A Mature foliage under side. Near R.H.S. Greyed-orange; 176B to 177A/B, yellow-green towards the tip; 147B/C. Outer tip greyed-purple; N186C, but slightly lighter, whole surface colour softened by an overall greyish hue. Leaf blade with an unclear (due to the greyish layer) greyed-purple reticulate variegation; N186B. Tip; 178B.
   Venation.—Type: Parallel. Venation coloration. Identical/Indistinguishable from foliage.
Petiole: Not present, sessile.
Flower: Flowering has not been observed, and is not commercially important in this cultivar.

### OTHER CHARACTERISTICS

Disease resistance: Neither resistance nor susceptibility to diseases or pests has been observed in this variety.
Drought tolerance and cold tolerance: Observed to tolerate temperatures to 35° C. without any negative effects. Temperature tolerance above this range has not been observed. Tolerance of cold temperatures have not been observed.
Fruit/seed production: Not observed.

What is claimed is:

1. A new and distinct cultivar of Vriesea plant named ‘Kiwi Cream’ as herein illustrated and described.
